(19) United States
(12) Patent Application Publication
(10) Pub. No.: US 2024/0298951 A1
(43) Pub. Date: Sep. 12, 2024

(54) DRY ELECTRODE DEVICE

(71) Applicant: Lohmann GmbH & Co. KG, Neuwied (DE)

(72) Inventors: Volker LUTZ, Rengsdorf (DE); Christian WEISENSTEIN, Kirchen (DE)

(21) Appl. No.: 18/596,191

(22) Filed: Mar. 5, 2024

(30) Foreign Application Priority Data
Mar. 7, 2023 (DE) 10 2023 000 860.5

(51) Int. Cl. A61B 5/259; A61B 5/268; A61B 5/28

(52) U.S. Cl. CPC A61B 5/259; C09J 9/02; C09J 133/064; A61B 5/28; C09J 2301/314; C09J 2301/408

(57) ABSTRACT

The present technology relates to a dry electrode device with at least one electrically conductive connection unit and with an, in particular electrically conductive, adhesive layer unit, which has a contact surface for contacting a skin and which is configured to provide an electrical energy transmission between the connection unit and the skin in an application state. To improve a design, it is proposed that, in a plan view, a contour area of the connection unit overlaps a contour area of the contact surface by at least 80%.

### CROSS-REFERENCE TO RELATED APPLICATIONS

The present application claims priority to German patent application DE 10 2023 000 860.5, “Trockenelektrodenvorrichtung” (Dry electrode device), filed Mar. 7, 2023.

### FIELD OF TECHNOLOGY

The present technology relates to a dry electrode device.

### BACKGROUND

Traditional gel electrodes have the disadvantage of drying out over time and are therefore not suited for long-term applications. Dry electrodes do not have this disadvantage, however, there is still a need for dry electrodes which are suitable for long-term applications, not significantly more expensive than other types of electrodes and, over the duration of the application, neither debond nor cause skin irritations.

Dry electrodes and electrically conductive adhesives used in the manufacturing of them are, for example, known from WO 2020/178217 A1. The adhesive disclosed comprises an ionic liquid to provide a sufficient electrical conductivity. The reason presented is that providing an electrical conductivity solely via electrically conductive particles would require an addition of so many electrically conductive particles that the bond of the adhesive would be influenced negatively. Furthermore, the electrodes disclosed comprise electrically conductive layers, which are arranged between the electrically conductive adhesive layer and a connection unit and may for example consist of silver/silver chloride. These electrically conductive layers enlarge and/or displace the area in which signals may be transferred through the electrically conductive adhesive to a connection unit, which may be a metallic snap fastener. This increases the quality of the signal and the freedom of design but increases both the material costs and the complexity of the manufacturing process.

It is an object of the present technology to provide a generic device with improved features relating to a design. This object is achieved by the features of claim 1, while other embodiments and improvements of the present technology may be taken from the dependent claims.

### BRIEF SUMMARY

The present technology relates to a dry electrode device with at least one electrically conductive connection unit and with a, in particular electrically conductive, adhesive layer unit, which has a contact surface for contacting a skin and which is configured to provide an electrical energy transmission between the connection unit and the skin in an application state.

### BRIEF DESCRIPTION OF FIGURES

FIG. 1 A schematic depiction of a dry electrode device in a cross-sectional view.

FIG. 2 A schematic depiction of the dry electrode device in a plan view from above.

FIG. 3 A schematic depiction of a further embodiment of a dry electrode device in a cross-sectional view.

FIG. 4 A schematic depiction of the dry electrode device shown in FIG. 3 in a plan view from above.

FIG. 5 A schematic depiction of a further embodiment of a dry electrode device in a cross-sectional view.

FIG. 6 A schematic depiction of the dry electrode device shown in FIG. 5 in a plan view from above.

### DESCRIPTION

It is proposed that in a plan view, a contour area of the connection unit overlaps a contour area of the contact surface by at least 80%. This way, the effective skin surface, through which signals can be transferred to the connection unit, is much smaller than in dry electrodes known from the state of the art, which may use silver/silver chloride layers to increase a size of the contact surface to be double or even triple the size of the connection unit. Unexpectantly, it has been found that the signal quality is still sufficient for a lot of applications even when using small contact surfaces. This increases a freedom of design and enables a cheaper and easier manufacturing of the dry electrode device, since additional electrically conductive layers and electrically conductive adhesive layers, which expand past the connection unit, may be omitted.

It is possible that the dry electrode device forms a part of a dry electrode system, wherein the dry electrode system comprises one or more dry electrode devices and additional functional components such as casings, cables, connectors, carrying straps, displays, processors and/or communication devices. The dry electrode device may also be embodied as a disposable dry electrode, which is configured to be applied on skin and disposed of after completion of a use period. This way, a compact and cheap design of the dry electrode device can be achieved.

It could be imagined that the dry electrode device comprises a multitude of connection units and/or adhesive layer units. In this case, either any one of the connection units provides an electrical energy transmission between the skin and the connection unit through one of the adhesive layer units, or a multitude of connection units provides an electrical energy transmission between the skin and the connection unit through a single adhesive layer unit. The dry electrode device may also comprise exactly one connection unit and exactly one adhesive layer unit. It could be imagined that the dry electrode device comprises at least one sensor unit in addition to the connection unit and the adhesive layer unit, wherein the sensor unit is configured to measure bio signals. The sensor unit could forward the measured bio signals either to the connection unit or to a separate further connection unit. For example, the sensor unit could comprise at least one optical sensor such as a PPG sensor, at least one heat sensor, at least one pH sensor and/or at least one moisture sensor.

It is possible that the dry electrode device comprises at least one cover unit. The cover unit is in particular configured to, together with the adhesive layer unit, fix the dry electrode device relative to the skin in the application state. The cover unit may also comprise a carrier, which could for example be embodied as a textile or a cellulose material, and a pressure-sensitive skin adhesive, which is arranged on the carrier. The cover unit may also be connected to the connection unit via a force—and/or form-locking connection. Also, in the plan view, a contour of the cover unit encompasses a contour of the adhesive layer unit, wherein the cover unit provides an adhesive bond with the skin within the encompassing area of the contour in the application state.

The size of the connection unit, adhesive layer unit and cover unit is in particular dependent on an application field of the dry electrode device. A contour area of the cover unit may have a surface area of at most 100 cm², and also at most 75 cm², and also at least 8 cm², and also at least 12 cm². The contour area of the adhesive layer unit may also have a surface area of at most 40 cm², and also at most 30 cm², and also of at least 2 cm², and also at least 4 cm² in the application state.

The dry electrode device comprises at least one release liner. The release liner may cover a side of the adhesive layer unit opposite of the connection unit and is configured to be removed before an application of the dry electrode device.

The connection unit may comprise any electrically conductive materials. “Electrically conductive materials” in this context are to be understood as all materials which exhibit an electrical conductivity of at least 10⁶ S/m at 25° C., in particular metals and carbon. It is possible that the connection unit consists entirely of electrically conductive materials. Alternatively, the connection unit could comprise a coating made of one or multiple electrically conductive materials. Furthermore, the connection unit could comprise electrically conductive particles, which comprise one or more electrically conductive materials.

The connection unit comprises a contact element, which is located on a side of the connection unit facing the adhesive layer unit and configured to provide an electrical energy transmission between the skin and the connection unit in the application state. Also, the contact element comprises a contact plate with a length and width may be five times, or ten times, or fifteen times its thickness. Also, the contact plate may comprise an even surface facing the adhesive layer unit. The contact plate may in particular form the contact element in its entirety, alternatively, the contact place may only form a part of the contact element. It could be imagined that the adhesive layer unit has a constant thickness and that the contact element is arranged on the adhesive layer unit. Alternatively, the adhesive layer unit could have a reduced thickness in the area near the contact element, wherein the contact element is partially or completely embedded in the adhesive layer unit.

The connection unit comprises a connection element which is arranged at a side of the connection unit opposite to the adhesive layer unit and configured to be connected to an external unit in the application state. The connection element can provide the connection to the external unit via any, in particular reversible, form—and/or force-locking connection types, for example, the connection element could be embodied as a snap element, clip element, thread element, hook element and/or dovetail joint element. The connection element is manually connectable to the external unit. Also, the contact element and the connection element together form a snap fastener element. The external unit may for example provide a forwarding and/or processing of bio signals and/or supplying of electrical impulses, for example for the stimulation of muscle groups.

It could be possible that the connection element and the contact element are embodied as parts of a single cohesive component. Alternatively, the connection unit may, in addition to the connection element and the contact element, comprise a coupling element which couples the connection element and the contact element to each other. The coupling element could couple the connection element and the contact element to each other via a, in particular reversible, form- and/or force-locking connection and be embodied as a snap element, clip element, thread element, hook element and/or dovetail joint element. Alternatively, the coupling element could couple the connection element and the contact element to each other via an irreversible form-locking connection and be embodied as a rivet or an adhesive. The connection element and the contact element may also comprise corresponding parts, via which the connection element and the contact element are coupled to each other. These parts could together form a, in particular reversible, form- and/or force-locked connection, for example a snap connection, clip connection, thread connection, hook connection and/or dovetail joint connection. The parts together may form an irreversible, form-locked connection, for example a rivet connection, adhesive connection and/or weld connection.

The adhesive layer unit can comprise any number of adhesive layers, which may be arranged on top of each other or on a carrier. In particular, the adhesive layer unit comprises at least one adhesive layer. It could be imagined that the adhesive layers of the adhesive layer unit comprise recesses and/or a thickness which changes locally. All adhesive layers of the adhesive layer unit may also be embodied as cohesive layers with a thickness which, except for a reduced thickness in the area near the contact element in the case that the contact element is embedded in the adhesive layer unit, is constant.

A “contact surface of the adhesive layer unit” is to be understood as a part of a surface of the adhesive layer unit opposite to the connection unit, wherein bio signals are transferred from the part of the skin in contact with the contact surface to the contact element via an electrical energy transmission in the application state. It could be imagined that the electrical energy transmission is a capacitive energy transmission and that the contact element is embodied as a capacitive electrode, wherein the adhesive layer unit only provides a capacitive electrical coupling between the connection unit and the skin in the application state. In this case, the adhesive layer unit may be devoid of electrically conductive materials, wherein the contact surface is dependent on a size of the contact element and a distance of the contact element from the skin in the application state.

The electrical energy transmission is an electrically conductive energy transmission and the contact element is electrically conductively coupled to the skin in the application state, wherein the adhesive layer unit provides a direct electrical connection between the connection unit and the skin in the application state. In this case, the adhesive layer unit may be isotropically electrically conductive, by which the contact surface and the surface of the adhesive layer unit opposite to the connection unit are identical. The adhesive layer unit may also be anisotropically electrically conductive, by which the size of the contact surface is identical to the surface of the contact element facing the adhesive layer unit.

The “application state” in this context is to be understood as a state in which the dry electrode device is attached to the skin and functional. In particular, the connection element is connected to the external unit in the application state, also the dry electrode device may measure bio signals, in particular a ECG signal, and forwards the bio signals to the external unit for processing.

The “plan view” is to be understood as a view of the dry electrode device with a line of sight aligned parallel to a surface normal of the adhesive layer unit. A “contour area” of an element or a unit is to be understood as an area encompassed in the plan view by a contour of the element or the unit. The contour of an element or a unit is to be understood as the smallest possible circumference encompassing the entirety of the element or the unit. A contour of the connection unit is identical to a contour of the contact element, particularly a contour of the contact plate. The contour of an element is to be considered while disregarding any elements which may be arranged over or under the element, therefore in the plan view of the dry electrode device, it is irrelevant whether the dry electrode device is viewed from above or from below.

It could be imagined that the contour of the adhesive layer unit and the contour of the connection unit have different shapes. The contour of the adhesive layer unit and the contour of the connection unit may have the same shape. The contour of the adhesive layer unit and the contour of the connection unit may be arranged concentric to each other. The contour of the adhesive layer unit and the contour of the connection unit may in particular have any shape, for example, the contours may be equivalent to the circumference of a rectangle, in particular circle, oval, in particular circle, or any other shape consisting of connected straight lines and arcs.

It is possible that the dry electrode device comprises electrically conductive layers, which are arranged between the adhesive layer unit and connection unit. In order to further simplify a design of the dry electrode device, it is proposed that the adhesive layer unit is arranged directly on the connection unit. The adhesive layer unit being arranged directly on the connection unit is to be understood as the adhesive layer unit and the contact element being devoid of electrically conductive coatings, carriers or covers which are partially or entirely arranged between the adhesive layer unit and the contact element. This way, by omitting a contact surface which is larger than the contact element, material costs can be save and manufacturing steps can be omitted.

It could be imagined that the contour area of the adhesive layer unit is smaller than or identical to the contour area of the connection unit. In this case, it would be necessary for the dry electrode device to comprise at least one additional layer which is arranged between the skin and the connection unit in the application state and which comprises a contour area bigger than the contour area of the connection unit in the plan view in order to prevent a direct electrical connection of the skin via the connection unit. Alternatively, the contour area of the adhesive layer unit could be a multitude bigger than the contour area of the connection unit, by which, for example, the cover unit could be omitted. In order to simplify a design of the dry electrode device, it is proposed that in the plan view, the contour area of the adhesive layer unit may be at most 100%, or at most 75%, or at most 50% or also at most 25% larger than the contour area of the connection unit. In the plan view, a minimal distance between the contour of the adhesive layer unit and the contour of the connection unit is at least 1 mm, or at least 2 mm at any point, by which a direct electrical connection of the skin via the connection unit can be prevented. This way, adhesive and, in case a cover unit is present, cover material can be saved. A skin area which is covered by the dry electrode device can be reduced, by which skin irritations can be prevented and the comfort of the wearer can be increased, even if the breathability of the adhesive layer unit stays the same.

In order to further simplify a design of the dry electrode device, it is proposed that the adhesive layer unit is configured to provide a complete fixation of the dry electrode device relative to the skin in the application state. In particular, the dry electrode device is devoid of cover units which contribute to a fixation of the dry electrode device relative to the skin in the application state. This way, material costs can be saved and manufacturing steps can be omitted. A more compact design of the dry electrode device may be realized, by which a comfort of the wearer may be further increased.

In order to further improve upon a design of the dry electrode device, it is proposed that the adhesive layer unit comprises at least one adhesive layer which comprises a polymer network with at least one acrylate copolymer and at least one type of electrically conductive particles and which is free of ionic liquids. A “polymer network” is to be understood as a superordinate structure which consists of linked polymers. It could be imagined that the polymer network comprises a multitude of different acrylate copolymers. The polymer network may comprise exactly one type of acrylate copolymer. The acrylate copolymer may in particular be embodied as a random copolymer, a block copolymer or a homo polymer. Just like the connection unit, the electrically conductive particles of the adhesive layer may also comprise any electrically conductive materials. The electrically conductive particles may consist entirely of the electrically conductive materials. For example, the electrically conductive particles may consist of an electrically conductive polymer such as PEDOT:PSS. Alternatively, the electrically conductive particles could be coated with electrically conductive materials. For example, the electrically conductive particles may comprise a plastic core, aluminum core or glass core, which is coated with copper, silver or other metals. The adhesive layer may exhibit an impedance of at most 50Ω at 10 Hz when measured vertically through the adhesive layer at 25° C.

Ther adhesive layer may be biocompatible. The adhesive layer being “biocompatible” is to be understood as the adhesive layer fulfilling the requirements of normed tests for the biocompatibility of substances. The normed tests are dependent on a country of application of the dry electrode device, the adhesive layer may also at least fulfill the requirements of a test for skin irritation according to ISO 10993-10, a test for skin sensitization according to ISO 10993-10 and a test for cytotoxicity according to ISO 10993-5.

The adhesive layer is embodied as a pressure-sensitive adhesive layer. A “pressure-sensitive adhesive layer” is to be understood as an adhesive layer which is permanently tacky at 25° C. and without a preceding activation step, for example a heating or an irradiation. The adhesive layer is devoid of structural adhesives configured to crosslink after an application, wherein the structural adhesives exhibit little to no tack before the crosslinking. The adhesive layer may also be placed directly on the skin in the application state. The adhesive layer may also embody the adhesive layer unit in its entirety. This way, a design of the dry electrode device can be further improved upon. It is noted that, by using an electrically conductive arylate adhesive a biocompatible adhesive layer suited for long-time application can be achieved. The previously mentioned unexpected revelation that a lower signal quality is still acceptable in many applications led to tests being conducted to see if the ionic liquids known from the state of the art can be omitted. These tests led to the realization that acrylate adhesives comprising electrically conductive particles can provide a sufficient signal quality coupled with a biocompatibility and a good long-time bonding on skin without the need for ionic liquids or electrically conductive salts.

Additionally, it is proposed that the acrylate copolymer comprises 70 to 98.5% by weight, or 85 to 96% by weight, or 90 to 95% by weight of non-polar acrylate monomers and 1.5 to 30% by weight, or 3 to 15% by weight, or 5 to 10% by weight of polar acrylate monomers, wherein in particular, the weight percentages add up to 100%. The terms “acrylate copolymer” and “acrylate monomer” are to be understood as encompassing both pure acrylate copolymers and acrylate monomers as well as methacrylate copolymers and methacrylate monomers. An acrylate monomer being “polar” in this context is to be understood as the acrylate monomer comprising at least one polar group, for example an OH group. An acrylate monomer being “non-polar” in this context is to be understood as the acrylate monomer being devoid of polar groups. In particular, any acrylate monomers known from the state of the art may be used, for example acrylic acid, methyl acrylate, ethyl acrylate, n-butyl acrylate, iso-butyl acrylate, tert-butyl acrylate, 2-ethylhexyl acrylate, 2-hydroxypropyl acrylate, 2-hydroxyethyl acrylate, iso-bornyl acrylate, N,N-dimethylaminoethyl acrylate, lauryl acrylate, stearyl acrylate, benzyl acrylate, methacrylic acid, methyl methacrylate, ethyl methacrylate, isopropyl acrylate, n-butyl methacrylate, iso-butyl methacrylate, tert-butyl methacrylate and/or cyclohexyl methacrylate. The acrylate copolymer may essentially consist of the polar and non-polar acrylate monomers. “Essentially” in this context is to be understood as the acrylate copolymer being able to comprise further components, such as remains of an initiator, however, these further components embody less than 0.5% by weight, or less than 0.1% by weight of the acrylate copolymer and do not affect the properties of the acrylate copolymer significantly. This way, a design of the dry electrode device can be further improved upon. An adjustment adjustment of the polarity of the acrylate copolymer can optimize a breathability of the adhesive layer. Insufficient breathability leads to an accumulation of moisture between the dry electrode device and the skin, which causes skin irritations and reduces the long-time bonding of the dry electrode device. Excessive breathability leads to an accumulation of moisture inside of the adhesive layer, which also reduces the long-time bonding of the dry electrode device.

The given weight percentages relate to a share of the respective substance in an adhesive mass which is coated in order to form the adhesive layer and goes through a polymerization reaction either before or after the coating. In particular, the adhesive layer may, in addition to the substances forming the polymer network after the polymerization reaction, comprise further substances, for example crosslinkers, tackifiers, antioxidants, reactive diluents, fillers such as for example glass spheres or ceramic spheres, foaming agents and further additives which are known to persons of ordinary skill in the art. The adhesive layer comprises at least 90% by weight, or at least 95% by weight, of substances of the acrylate copolymer.

The phrasings “X to Y”, “at least X”, “at most X” in this context are to be understood as including the borders of the respective ranges.

It could be imagined that the acrylate copolymer is devoid of acrylic acid and instead exclusively comprises other types of polar acrylate monomers such as for example 2-hydroxyethyl acrylate. In order to further simplify a design of the dry electrode device, it is proposed that the acrylate copolymer comprises 0.1 to 10% by weight, or 3 to 7% by weight of acrylic acid. Assigning a weight percentage relating to the acrylate copolymer is to be understood as the corresponding substances forming a part of the acrylate copolymer after the polymerization reaction. The acrylic acid may be the sole polar acrylate monomer of the acrylate copolymer. This way, a glass transition temperature of the acrylate copolymer can be optimized. The high glass transition temperature of the acrylic acid, which is 100.85° C., may be used to adjust the glass transition temperature of the acrylate copolymer towards a higher temperature. Also, through adjusting the glass transition temperature of the acrylate copolymer, a tackiness and a cohesion of the adhesive layer can be optimized, by which a premature debonding of the dry electrode due to a cohesive failure and a presence of leftover residue on the skin can be prevented. Excessive cohesion of the adhesive layer leads to an insufficient wetting of the skin due to the uneven skin surface. Insufficient cohesion of the adhesive layer leads to an insufficiently strong bond between the dry electrode device and the skin, which may cause displacement or partial to complete debonding of the dry electrode device, furthermore, an insufficient cohesion of the adhesive layer may lead to leftover residue being present on the skin after a removal of the dry electrode device after the use period.

Optimization of a property of the acrylate copolymer via a specific selection of acrylate monomers is to be understood as the acrylate copolymer, which is created via a polymerization reaction, exhibiting different properties than a comparable acrylate copolymer which is created via the same polymerization reaction but comprises different acrylate monomers or a different distribution of the same acrylate monomers. The polymerization reaction being the same is to be understood as a number of monomer units which are linked during the polymerization reaction to form the acrylate polymer being identical for both polymerization reactions. For example, an acrylate copolymer formed out of short-chain monomers is by nature lighter than an acrylate copolymer formed out of long-chain monomers. This may be usefule in industrial scale manufacturing processes, since the facilities used in these processes place certain requirements on the polymerization reaction, in particular concerning the reaction speed, by which an adjustment of the properties of the created copolymers via an adjustment of the polymerization reaction is often not possible.

Furthermore, it is proposed that the acrylate copolymer comprises at least 50% by weight, or at least 70% by weight, or at least 90% by weight of acrylic acid esters whose molar masses are at least 100 g/mol, or at least 120 g/mol. For example, the acrylic acid esters may comprise n-butyl acrylate, 2-ethylhexyl acrylate, isobutyl acrylate, ethyl acrylate and/or propyl acrylate. This way, an average molar mass distribution of the acrylate copolymer can be optimized. An average molar mass distribution of the acrylate copolymer is 450.000 to 900.000 g/mol, or 500.000 to 800.000 g/mol or also 600.000 to 700.000 g/mol. The cohesion and viscosity of the adhesive layer may be optimized via the adjustment of the average molar mass distribution of the acrylate copolymer, in particular, the adhesive used for manufacturing of the adhesive layer may be coated with generic coating processes such as for example curtain coating, blade coating or screen coating. It could also be imagined that the adhesive is applied via ink jet printing processes or 3D printing processes.

In addition, it is proposed that the adhesive layer comprises 5 to 30% by weight, or 10 to 25% by weight, or 15 to 20% by weight of electrically conductive particles. Tests have revealed that the addition of electrically conductive particles in the amount stated provides a sufficient electrical conductivity without negatively affecting the long-time bonding of the adhesive layer.

It could be imagined that the electrically conductive particles are embodied as metal particles or metal-coated particles. In order to further improve upon a design of the dry electrode device, it is proposed that the electrically conductive particles are embodied as carbon particles. In particular, the electrically conductive particles may be embodied as graphite particles or graphene particles. This way, a sufficient electrical conductivity of the adhesive layer may be realized while keeping the amount of added electrically conductive particles low. Tests have revealed that electrically conductive particles comprising metals either do not provide a sufficient electrical conductivity or have to be added in large amounts that negatively affect the long-time bonding of the adhesive layer. Only carbon particles provided results that fulfilled the both the requirement regarding the signal quality and the requirement regarding a long-time bonding of the dry electrode device.

Generally, the electrically conductive particles may have any shape and size, for example, they may be shaped like spheres, rods or have an amorphous shape. In order to further improve upon a design of the dry electrode device, it is proposed that the electrically conductive particles are embodied as micro-platelets. “Micro-platelets” are to be understood as particles in the shape of layers with an average diameter of at least 1 μm, or at least 3 μm, and at most 20 μm, or at most 10 μm. The average diameter, which is also known as the “lateral size”, is a manufacturer specification defining the average particle size. A thickness of the micro-platelets is at least 1 nm, or at least 5 nm, or at most 500 nm, or at most 250 nm. This way, a sufficient electrical conductivity, biocompatibility, and long-term bonding of the dry electrode device can be achieved. The low thickness and large surface area of the micro-platelets provides a high contribution to the electrical conductivity of the adhesive layer for each electrically conductive particle, which allows for a reduction of the amount of electrically conductive particles which have to be added. As a result, nanoparticles which have a diameter on the nanometer scale in any direction and are thus able to enter human cells can be omitted.

Additionally, it is proposed that a thickness of the adhesive layer, in particular outside of the area near the contact element, is 10 to 100 μm, or 20 to 80 μm, or 30 to 60 μm. This way, a design of the dry electrode device may be further improved upon. Tests have revealed that a thickness of the adhesive layer within the proposed range achieves optimal properties regarding an electrical conductivity and long-term bonding. Insufficient thickness of the adhesive layer leads to easy tearing, which makes the adhesive layer hard to process and does not allow for a strong bond. Excessive thickness of the adhesive layer leads to high material costs both regarding the adhesive as well as regarding the electrically conductive particles, and due to the high thickness, there is a high likelihood of cohesive failure, furthermore, the amount of electrically conductive particles which would have to be added would be so high that a long-term bonding of the adhesive layer would be negatively affected.

Furthermore, it is proposed that a glass transition temperature of the adhesive layer is −50 to −20° C. Tests have revealed that an adhesive layer having a glass transition temperature within the proposed range achieves optimal properties regarding a cohesion. The adhesive layer may also have a storage module of at least 0.1 MPa and therefore fulfils the Dahlquist criterion. This way, a sufficient wetting of the skin by the adhesive layer can be achieved and a long-time bonding of the dry electrode device can be improved.

It could be imagined that the adhesive layer comprises more than 10% by weight of crosslinkers in order to optimize a cohesion of the adhesive layer. In order to further improve upon a design of the dry electrode device, it is proposed that the adhesive layer comprises 0.1 to 10% by weight, or 0.1 to 5% by weight, or 0.15 to 1% by weight of crosslinkers. A “crosslinker” is to be understood as a substance configured to link individual acrylate copolymers after the polymerization reaction in order to form the polymer network. In particular, crosslinkers differ from “curing agents” which are configured to link the acrylate monomers during the polymerization reaction in order to form the acrylate copolymer. In particular, the crosslinker may be added before, during or after a coating of the adhesive layer. The crosslinkers may in particular be any known crosslinkers for acrylate adhesives, such as for example ethylenglycol diacrylate, N,N′-methylenebisacrylamide or aluminum acetylacetonate. It could be imagined that the adhesive layer comprises multiple different types of crosslinkers. The adhesive layer comprises one type of crosslinker. This way, a long-term bonding of the dry electrode device can be improved upon. Adjusting the cohesion via the glass transition temperature and the average molar mass distribution of the acrylate copolymer removes the need to adjust the cohesion via the addition of crosslinkers. Also, the adhesion of the adhesive layer can be improved upon via a reduction of the required amount of crosslinkers, since crosslinkers reduce the number of free copolymer chains which contribute to creating a bond to the skin.

In a further aspect of the technology, it is proposed that the adhesive layer unit comprises a first adhesive layer and a second adhesive layer. The first adhesive layer is arranged directly on the connection unit, in particular the contact element. Also, the second adhesive layer is placed directly on the skin in the application state. In particular, the first adhesive layer and the second adhesive layer each comprise an electrically conductive adhesive. The first adhesive layer and the second adhesive layer are both devoid of structural adhesives configured to crosslink after an application, wherein the structural adhesives exhibit little to no tack before the crosslinking. In particular, the first adhesive layer and the second adhesive layer comprise pressure-sensitive adhesives. For example, the first adhesive layer and the second adhesive layer could each comprise a synthetic-rubber-based pressure-sensitive adhesive, a natural-rubber-based pressure-sensitive adhesive, an acrylate-based pressure-sensitive adhesive, a polyurethane-based pressure-sensitive adhesive or a silicone-based pressure-sensitive adhesive. It could be imagined that the first adhesive layer and the second adhesive layer comprise adhesives which are identical to each other. The first adhesive layer and the second adhesive layer comprise adhesives with are different to each other. Generally, the first adhesive layer and the second adhesive layer could have the same thickness, alternatively, the first adhesive layer and the second adhesive layer could have different thicknesses. It could be imagined that in the plan view, a contour of the first adhesive layer is a different shape than a contour of the second adhesive layer, also, in the plan view, the contour of the first adhesive layer and the contour of the second adhesive layer have the same shape. The second adhesive layer may also comprise a biocompatible adhesive. This way, a design of the dry electrode device, in particular regarding a freedom of design, can be improved upon. As compared to embodiments of the adhesive layer unit comprising only a single adhesive layer, an extended spectrum of applications can be served. For example, applications on sensitive and/or thin skin may require a reduction of the adhesive force in order to prevent skin irritations and/or skin damages. However, these applications still require a strong fixation of the connection unit in order to ensure a good signal quality. Therefore, these applications make it impossible to satisfy all the requirements with just a single adhesive layer.

In order to further improve upon a design of the dry electrode device, it is proposed that the first adhesive layer has an adhesive force on metal according to DIN EN 1939 (December 2003) of at least 10 N/25 mm, or at least 15 N/25 mm, or at least 25 N/25 mm and the second adhesive layer has an adhesive force on skin according to DIN EN 1939 (December 2003) of at least 1 N/25 mm, or at least 2 N/25 mm or at least 5 N/25 mm. This way, a signal quality can be improved and at the same time, skin irritations and/or skin damages caused by a removal of the dry electrode device can be prevented.

The adhesive force according to DIN EN 1939 (December 2003) is defined as the force required to remove an adhesive strip from a specific substrate, at a specific angle and at a specific speed. Measurement of the adhesive force according to DIN EN 1939 (December 2003) is performed as follows: A sample of the adhesive layer with a width of 25 mm and a length of 100 mm is manufactured. Afterwards the sample is applied on a substrate, resulting in a bonded surface of 625 mm². For the first adhesive layer, the substrate consists of steel, for the second adhesive layer, the substrate is meant to emulate human skin and may consist of a polyurethane leather or an artificial skin. On a side of the sample opposite to the substrate, a roughly 10 cm long strip of a 50 μm thick PET foil is applied. Afterwards, the sample is pressed to the substrate using a 5 kg roller in order to ensure a complete bonding. After 10 minutes, the sample is removed from the substrate at a speed of 300 mm/min and at an angle of 180°, wherein the force required to completely remove the sample is measured. The median of five measurements then defines the adhesive force in N/25 mm. The measurement is made under standard conditions which are defined as a temperature of 23° C.±2° C. and a humidity of 50%±5%.

It could be imagined that the first adhesive layer and the second adhesive layer are laminated onto each other directly. The adhesive layer unit may comprise an electrically conductive carrier which is arranged between the first adhesive layer and the second adhesive layer. It is possible that the electrically conductive carrier is embodied as a textile and consists of metal fibers, coated glass fibers, coated plastic fibers and/or electrically conductive polymer fibers. The electrically conductive carrier may be devoid of recesses and can be embodied as, for example, a metal foil, an electrically conductive polymer foil or a carbon foil. This way, a freedom of design can be further improved upon. The first adhesive layer and the second adhesive layer may comprise adhesives which are difficult to bond with each other. Also, the first adhesive layer may comprise adhesives which are non-biocompatible as long as the electrically conductive carrier provides a sufficient barrier to prevent diffusion of the adhesive.

Generally, the first adhesive layer and the second adhesive layer may have the same shape and size. In order to further improve upon a design of the dry electrode device, it is proposed that in the plan view, a contour area of the second adhesive layer is at least 25%, or at least 50% larger than a contour area of the first adhesive layer. In particular, the electrically conductive carrier provides an extension of the contact surface, wherein in the plan view, a contour of the contact surface is identical to a contour of the second adhesive layer. In particular, the second adhesive layer defines a size of the contact surface. The contour of the first adhesive layer and the contour of the second adhesive layer are arranged concentric to each other. Inm the plan view, a minimal distance between the contour of the second adhesive layer and the contour of the first adhesive layer at each point is at least 1 mm, or at least 2 mm. This way, an asymmetrical design of the dry electrode device can be realized, which ensures that, should the first adhesive layer comprise non-biocompatible adhesives, a sufficient distance between an edge of the first adhesive layer and an edge of the second adhesive layer to prevent a diffusion of the adhesive of the first adhesive layer can be ensured.

Additional aspects are disclosed in the following description of the figures.

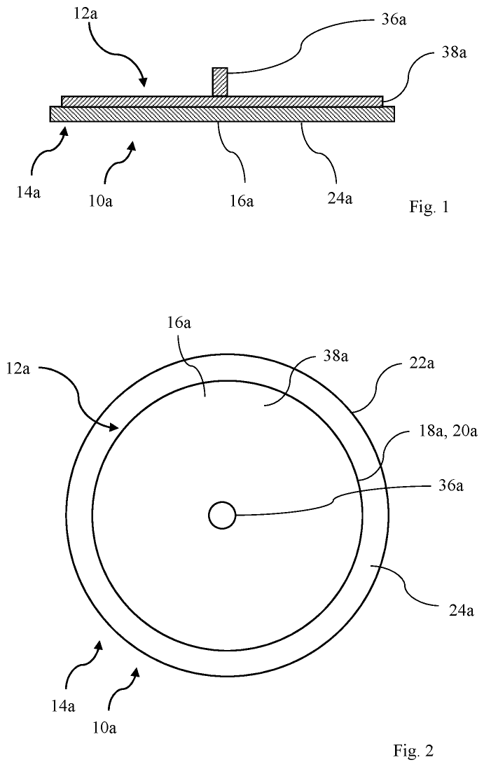

FIGS. 1 and 2 show a dry electrode device 10a. The dry electrode device 10a is embodied as a singular, disposable dry electrode configured to be placed on a skin in an application state, to measure an ECG signal and to forward the collected data to an external unit for processing. Alternatively, the dry electrode device 10a could be part of a dry electrode system (not shown) comprising multiple dry electrode devices. Furthermore, the dry electrode device 10a could, in addition to or instead of the ECG signal, measure other bio signals. The dry electrode device 10a is configured to measure the ECG signal based on a direct electrical connection, alternatively, the dry electrode device 10a could be configured to measure the ECG signal based on an indirect, capacitive electrical coupling.

The dry electrode device 10a comprises an electrically conductive connection unit 12a. The connection unit 12a consists of stainless steel. Alternatively, the connection unit 12a could consist of a plastic coated with electrically conductive materials or comprising electrically conductive particles. The connection unit 12a comprises a connection element 36a. The connection element 36a is embodied as a snap fastener element and configured to provide an electrically conductive snap connection with the external unit in the application state. Alternatively, the connection element 36a may be embodied as any other connection element, such as for example a clip element or a thread element.

The connection unit 12a comprises a contact element 38a. The contact element 38a is embodied as a contact plate. The contact element 38a and the connection element 36a are embodied as a single cohesive component. Alternatively, the contact element 38a and the connection element 36a could be connected to each other via any connection, such as for example a rivet connection, adhesive connection, weld connection, snap connection or screw connection. In particular, the contact element 38a and the connection element 36a may together form a snap fastener element in an alternative embodiment.

The dry electrode device 10a comprises an electrically conductive adhesive layer unit 14a. The electrically conductive adhesive layer unit 14a comprises exactly one adhesive layer 24a. Alternatively, the electrically conductive adhesive layer unit 14a may comprise any number of further adhesive layers. The adhesive layer unit 14a is arranged directly on the connection unit 12a. The contact element 38a is arranged on the adhesive layer 24a. Alternatively, the contact element 38a may be partially or completely embedded in the adhesive layer 24a.

The adhesive layer unit 14a comprises a contact surface 16a for contacting the skin. The contact surface 16a is a partial surface of a surface of the adhesive layer unit 14a opposite to the connection unit 12a. The adhesive layer unit 14a provides an electrical connection between the connection 12a and the skin in the application state. The electrical connection is a direct electrical connection through the adhesive layer 24a in the area of the contact surface 16a.

The adhesive layer unit 14a is configured to provide a complete fixation of the dry electrode device 10a relative to the skin in the application state. Alternatively, the dry electrode device 10a could additionally comprise a cover unit (not shown) consisting of a textile and a pressure-sensitive adhesive and extending past the adhesive layer unit 14a in order to contribute to a fixation of the dry electrode device 10a relative to the skin in the application state. The dry electrode device 10a is shown in an application state. The dry electrode device 10a comprises a release liner (not shown) in a non-applied state, wherein the release liner covers a side of the adhesive layer unit 14a opposite to the connection unit 12a.

FIG. 2 shows the dry electrode device 10a in a plan view from above. In the plan view, contours of elements and units are to be considered while disregarding any further elements and units which may be arranged over or under the respective element and unit, therefore, the contours are unaffected by whether a viewpoint of the plan view is from above or from below. A contour 22a of the adhesive layer unit 14a and a contour 20a of the connection unit 12a have the same shape and are arranged concentrical to each other. The contour 22a of the adhesive layer unit 14a and the contour 20a of the connection unit 12a are circular, alternatively, the contour 22a of the adhesive layer unit 14a and the contour 20a of the connection unit 12a may have any other shape, such as for example a rectangular or oval shape. The contour 22a of the adhesive layer unit 14a encompasses the contour 20a of the connection unit 12a, wherein a minimal distance between the contour 22a of the adhesive layer unit 14a and the contour 20a of the connection unit 12a at any point is 2 mm. A contour 18a of the contact surface 16a is identical to the contour 20a of the connection unit 12a, therefore a contour area of the connection unit 12a overlaps a contour area of the contact surface 16a for 100%. Alternatively, the adhesive layer 24a may be isotropically electrically conductive or the dry electrode 10a may comprise an electrically conductive layer arranged between the adhesive layer unit 13a and the connection unit 12a. In this case, the contour 18a of the contact surface 16a would be identical to the contour 22a of the adhesive layer unit 14a, wherein the contour area of the connection unit 12a would be smaller than the contour area of the contact surface 16a and would only overlap it by roughly 83%, since a contour area of the adhesive layer unit 14a is roughly 17% bigger than the contour area of the connection unit 12a.

The adhesive layer 24a comprises a polymer network with at least one acrylate copolymer and at least one type of electrically conductive particles. Alternatively, it could be imagined that the adhesive layer 24a comprises an electrically conductive silicon-based pressure-sensitive adhesive and/or an electrically conductive rubber-based pressure-sensitive adhesive. The acrylate copolymer comprises 95% by weight of non-polar acrylate monomers and 5% by weight of polar acrylate monomers. The acrylate copolymer comprises 95% by weight of acrylic acid esters with an average molar mass of at least 100 g/mol. An average molar mass of the acrylate copolymer is 650.000 g/mol.

The adhesive layer 24a is anisotropically electrically conductive. The adhesive layer 24a comprises 17.5% by weight of electrically conductive particles. The electrically conductive particles are embodied as graphene micro-platelets. It could also be imagined that the electrically conductive particles are embodied as metallic micro-platelets or carbon micro-spheres. According to manufacturer specifications, the graphene micro-platelets have an average diameter of 5 μm and a thickness of below 50 nm.

The adhesive layer 24a has a thickness of 30 μm. A glass transition temperature of the adhesive layer 24a is −47° C. The adhesive layer 24a comprises 0.2% by weight of crosslinkers and 99.8% by weight of substances belonging to the acrylate copolymer. The following table shows a composition of the adhesive layer 24a:

| Substance | Function | Weight percentage |
|---|---|---|
| N-butyl acrylate | Non-polar acrylate | 39.9% by weight |
| | monomer | |
| 2-ethylhexyl | Non-polar acrylate | 54.9% by weight |
| acrylate | monomer | |
| Acrylic acid | Polar acrylate | 5% by weight |
| | monomer | |
| Aluminum | Crosslinker | 0.2% by weight |
| acetylacetonate | | |

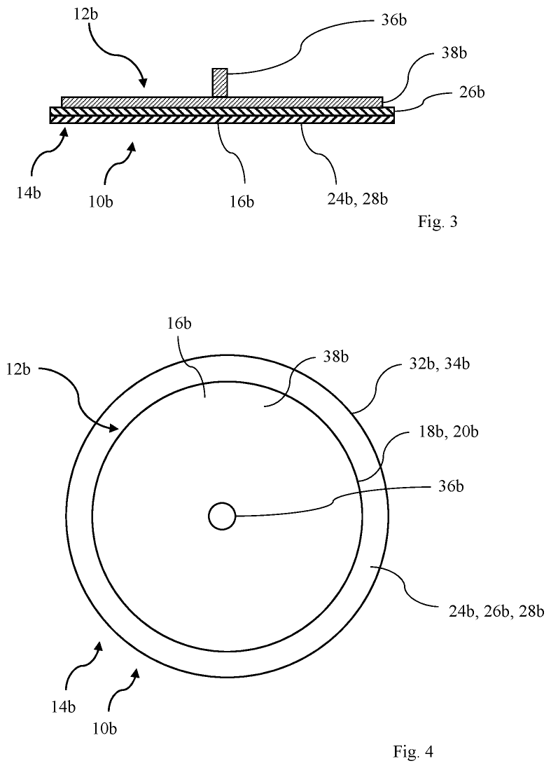

FIGS. 3 and 4 show an alternative embodiment of a dry electrode device 10b. In the following, only differences between this dry electrode device 10b and the dry electrode device 10a of the previous embodiment, in particular the embodiment of FIGS. 1 and 2, will be discussed.

The dry electrode device 10b comprises an adhesive layer unit 14b. The adhesive layer unit 14b comprises a first adhesive layer 26b and a second adhesive layer 28b. The first adhesive layer 26b and the second adhesive layer 28b both have a thickness of 15 μm. The first adhesive layer 26b is arranged directly on a connection unit 12b. The second adhesive layer 28b is placed directly on the skin in the application state. The first adhesive layer 26b has an adhesive force on metal according to DIN EN 1939 (December 2003) of at least 10 N/25 mm. The first adhesive layer 26b comprises an electrically conductive polyurethane-based pressure-sensitive adhesive. Alternatively, the first adhesive layer 26b could comprise any other electrically conductive pressure-sensitive adhesive configured to be used on metal surfaces.

The second adhesive layer 28b has an adhesive force on skin according to DIN EN 1939 (December 2003) of at least 2 N/25 mm. The second adhesive layer 28b is, except for its thickness, identical to the adhesive layer 24a of FIGS. 1 and 2, which is why the second adhesive layer 28b is assigned both the reference sign 24b and 28b in FIGS. 3 and 4. Alternatively, the second adhesive layer 26b could comprise any other electrically conductive pressure-sensitive adhesive configured to be used on skin surfaces.

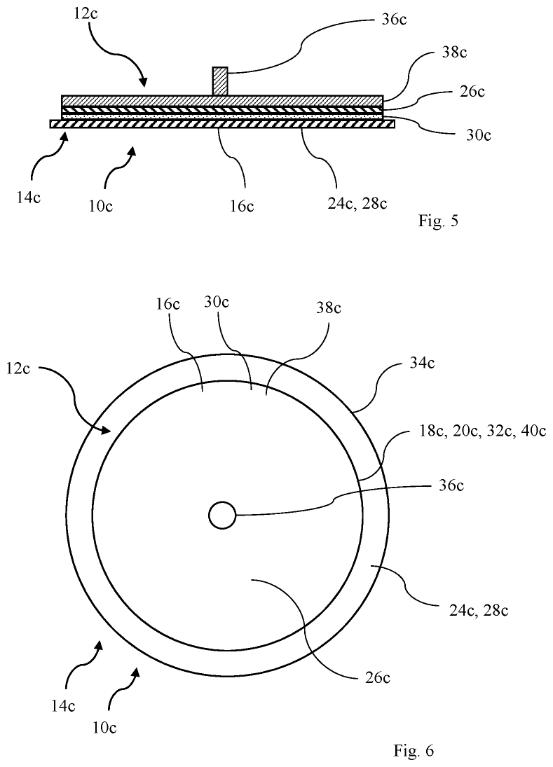

FIGS. 5 and 6 show an alternative embodiment of a dry electrode device 10c. In the following, only the differences between this dry electrode device 10c and the dry electrode device 10b of the previous embodiments, in particular the embodiment of FIGS. 3 and 4, will be discussed.

The dry electrode device 10c comprises an adhesive layer unit 14c. The adhesive layer unit 14c comprises an electrically conductive carrier 30c. The electrically conductive carrier 30c is arrange between the first adhesive layer 26c and the second adhesive layer 28c. The electrically conductive carrier 30c is embodied as an aluminum foil. Alternatively, the electrically conductive carrier 30c could be embodied as any other electrically conductive layer. In the plan view, a contour area of the second adhesive layer 28c is roughly 30% larger than a contour area of the first adhesive layer 26c. A contour 40c of the electrically conductive carrier 30c is identical to a contour 18c of a contact surface 16a of the adhesive layer unit 14c, a contour 20c of a connection unit 12c and a contour 32c of the first adhesive layer 26c. A contour 34c of the second adhesive layer 28c encompasses the contour 40c of the electrically conductive carrier 30c.

1. A dry electrode device with at least one electrically conductive connection unit and with an electrically conductive, adhesive layer unit, which has a contact surface for contacting skin and which is configured to provide an electrical energy transmission between the connection unit and the skin in an application state, characterized in that, in a plan view, a contour area of the connection unit overlaps a contour area of the contact surface by at least 80%.

2. The dry electrode device according to claim 1, wherein the adhesive layer unit is arranged directly on the connection unit.

3. The dry electrode device according to claim 1 wherein the plan view, a contour area of the adhesive layer unit is at most 100% larger than the contour area of the connection unit.

4. The dry electrode device according to claim 1, wherein the adhesive layer unit is configured to provide a complete fixation of the dry electrode device relative to the skin in the application state.

5. The dry electrode device according to claim 1, wherein the adhesive layer unit comprises at least one adhesive layer which comprises a polymer network with at least one acrylate copolymer and at least one type of electrically conductive particles and which is free of ionic liquids.

6. The dry electrode device according to claim 5, wherein the acrylate copolymer comprises 70 to 98.5% by weight of non-polar acrylate monomers and 1.5 to 30% by weight of polar acrylate monomers.

7. The dry electrode device according to claim 5, wherein the acrylate copolymer comprises 0.1 to 10% by weight of acrylic acid.

8. The dry electrode device according to claim 5, wherein the acrylate copolymer comprises at least 50% by weight of acrylic acid esters whose molar masses are at least 100 g/mol.

9. The dry electrode device according to claim 5, wherein the adhesive layer comprises 5 to 30% by weight of electrically conductive particles.

10. The dry electrode device according to claim 5, wherein the electrically conductive particles embodied as carbon particles.

11. The dry electrode device according to claim 5, wherein the electrically conductive particles are embodied as micro-platelets.

12. The dry electrode device according to claim 5, wherein a thickness of the adhesive layer is 10 to 100 μm.

13. The dry electrode device according to claim 5, wherein a glass transition temperature of the adhesive layer is −50 to −20° C.

14. The dry electrode device according to claim 5, wherein the adhesive layer comprises 0.01-10% by weight of crosslinkers.

15. The dry electrode device according to claim 1, wherein the adhesive layer unit comprises a first adhesive layer and a second adhesive layer.

16. The dry electrode device according to claim 15, wherein the first adhesive layer has an adhesive force on metal according to DIN EN 1939 (December 2003) of at least 10 N/25 mm and the second adhesive layer has an adhesive force on skin according to DIN EN 1939 (December 2003) of at least 1 N/25 mm.

17. The dry electrode device according to claim 15, wherein the adhesive layer unit comprises an electrically conductive carrier which is arranged between the first adhesive layer and the second adhesive layer.

18. The dry electrode device according to claim 17, wherein, in the plan view, a contour area of the second adhesive layer is at least 25% larger than a contour area of the first adhesive layer.
